(19) United States
(12) Patent Application Publication
(10) Pub. No.: US 2007/0288003 A1
(43) Pub. Date: Dec. 13, 2007

(54) LOCKING DEVICE AND METHOD, FOR USE IN A BONE STABILIZATION SYSTEM, EMPLOYING A BREAK-AWAY INTERFACE MEMBER RIGIDLY COUPLED TO A SEATING MEMBER

(76) Inventors: Jonathan M. Dewey, Memphis, TN (US); Christopher M. Patterson, Olive Branch, MS (US); Andrew R. Tuthill, Cardova, TN (US)

Correspondence Address: HESLIN ROTHENBERG FARLEY & MESITI P.C., 5 COLUMBIA CIRCLE, ALBANY, NY, 12203, US

(21) Appl. No.: 11/420,911

(22) Filed: May 30, 2006

(51) Int. Cl. A61F 2/30

(52) U.S. Cl. 606/61

(57) ABSTRACT

A locking device employing a break away interface member rigidly coupled to a seating member for use in securing a stabilization member within a bone stabilization system. The bone stabilization system includes a bone anchor, a stabilization member, a coupling mechanism and a locking device. The locking device is structured to engage the coupling mechanism and the coupling mechanism is configured to couple the stabilization member to the bone anchor. The locking device, which is configured to thread into the coupling mechanism, includes an interface member rigidly connected to the seating member. The interface member breaks away from the seating member at a break-off junction to drop into contact with the stabilization member and to secure the stabilization member in the coupling mechanism following continued engagement of the seating member into the coupling mechanism.

### CROSS-REFERENCE TO RELATED APPLICATIONS

This application contains subject matter which is related to the subject matter of the following applications, which are hereby incorporated herein by reference in their entirety:

“Multi-Axial Bone Attachment Assembly”, Coates et al., U.S. Ser. No. 10/870,011, filed Jun. 17, 2004, and published on Dec. 22, 2005 as Patent Application Publication No. US 2005/0283157 A1;

“Coupling Assemblies for Spinal Implants”, Justis et al., U.S. Ser. No. 11/197,799, filed Jan. 31, 2006;

“Force Limiting Coupling Assemblies for Spinal Implants”, Justis et al., U.S. Ser. No. 11/112,221, filed Jan. 31, 2006;

“Bone Anchor System Utilizing a Molded Coupling Member for Coupling a Bone Anchor to a Stabilization Member and Method Therefor”, Dewey et al., U.S. Ser. No. 11/414,878, filed May 1, 2006, (Attorney Docket No. P23147.00);

“Locking Device and Method, for Use in a Bone Stabilization System, Employing a Set Screw Member and Deformable Saddle Member”, Jeffrey Moore, U.S. Ser. No. 11/414,879, filed May 1, 2006, (Attorney Docket No. P24289.00); and

“Locking Device and Method Employing a Posted Member to Control Positioning of a Stabilization Member of a Bone Stabilization System”, Carls et al., U.S. Ser. No. ______, co-filed herewith, (Attorney Docket No. P25103.00).

### TECHNICAL FIELD

The present invention relates generally to orthopaedic implants used for the correction of spinal injuries or deformities, and more specifically, but not exclusively, concerns apparatuses and methods for fixing a particular segment or level of the spine, to allow for deformity correction or healing thereof.

### BACKGROUND OF THE INVENTION

In the field of spinal surgery, it is known to place implants into vertebrae for a number of reasons, including: (a) correcting an abnormal curvature of the spine; (b) to maintain appropriate vertebral spacing and provide support for broken or otherwise injured vertebrae; and (c) to perform other treatments in the spinal column.

Typical spinal implant or bone stabilization systems utilize a rod as the support and stabilizing element. In such a system, a series of two or more bone fasteners are inserted into two or more vertebrae to be instrumented. A rod or other stabilizing device is then placed within or attached to the head(s) of the bone fastener(s), or is placed within a coupling device that links the rod and the head(s) of the bone fastener(s). The connections between these multiple components are then secured, thereby fixing the supporting construct to multiple levels in the spinal column.

To advance the state of orthopaedic implants, enhancement to such bone stabilization systems are believed desirable, and are addressed herein.

### SUMMARY OF THE INVENTION

Briefly summarized, the present invention comprises in one aspect a locking device for use in a bone stabilization system. The bone stabilization system includes a bone anchor, a coupling mechanism and a stabilization member. The coupling mechanism is configured to couple the stabilization member to the bone anchor. The locking device includes an insertion head member, a seating member and an interface member. The seating member is integrally connected to the insertion head member at a break-off junction. The seating member is operatively associated with the coupling mechanism for securing the stabilization member within the coupling mechanism. The interface member is coupled to the seating member and is configured for disposition between the seating member and the stabilization member when the locking device is employed to secure the stabilization member within the coupling mechanism. Further, the interface member connects to the seating member at a break-off junction, thereby allowing the interface member to break away from the seating member and contact the stabilization member with engagement of the seating member to the coupling mechanism. Continued threading of the seating member into the coupling mechanism secures the stabilization member within the coupling mechanism with the interface member disposed between the seating member and the stabilization member. Following securement of the stabilization member within the coupling mechanism, the insertion head member breaks away from the seating member at the break-off junction when a pre-selected torque amount is applied to the insertion head member by a torque producing tool.

In another aspect, a bone stabilization system is provided which includes a bone anchor, a stabilization member and a coupling mechanism. The coupling mechanism is configured to operatively connect the bone anchor and the stabilization member. The bone stabilization system further includes a locking device which operatively connects to the coupling mechanism to secure the stabilization member within the coupling mechanism. The locking device includes an insertion head member, a seating member and an interface member. The seating member is connected to the insertion head member at a first break-off junction with at least one hole extending therethrough. The seating member is operatively associated with the coupling mechanism for securing the stabilization member within the coupling mechanism. The interface member is coupled to the seating member and is configured for disposition between the seating member and the stabilization member when the locking device is employed to secure the stabilization member within the coupling mechanism. Further, the interface member is rigidly connected to the seating member at a second break-off junction, thereby allowing the interface member to separate from the seating member with engagement of the seating member to the coupling mechanism. Following the breaking away of the interface member from the seating member, the interface member and the seating member remain rotatably coupled by the at least one post that is configured for disposition between the interface member and the seating member. Continued threading of the seating member into the coupling mechanism secures the stabilization member within the coupling mechanism with the interface member disposed between the seating member and the stabilization member. Upon achieving adequate securement of the stabilization member within the coupling mechanism, the insertion head member breaks away from the seating member at the first break-off junction when a pre-selected torque amount is applied to the insertion head member by a torque producing tool.

In another aspect, a bone stabilization system is provided which includes a bone anchor, a stabilization member and a coupling mechanism. The coupling mechanism is configured to operatively connect the bone anchor and the stabilization member. The bone stabilization system further includes a locking device which operatively connects to the coupling mechanism to secure the stabilization member within the coupling mechanism. The locking device includes an insertion head member, a seating member and an interface member. The seating member is connected to the insertion head member at a break-off junction with at least one hole extending therethrough. The seating member is operatively associated with the coupling mechanism for securing the stabilization member within the coupling mechanism. At least one post contained within the at least one hole and is configured for disposition between the seating member and the interface member. The at least one post rigidly couples the interface member to the seating member and is configured for disposition between the seating member and the stabilization member when the locking device is employed to secure the stabilization member within the coupling mechanism. Further, the at least one post is configured to include a break-off line, thereby allowing the interface member to contact the stabilization member with engagement of the seating member to the coupling mechanism. Continued threading of the seating member into the coupling mechanism secures the stabilization member within the coupling mechanism with the interface member disposed between the seating member and the stabilization member. When adequate securement of the stabilization member within the coupling mechanism is achieved, the insertion head member may be broken away from the seating member at the break-off junction by applying a pre-selected torque amount to the insertion head member with a torque producing tool.

In another aspect, a bone stabilization system is provided which includes a bone anchor, a stabilization member and a coupling mechanism. The coupling mechanism is configured to operatively connect the bone anchor and the stabilization member. The bone stabilization system further includes a locking device which operatively connects to the coupling mechanism to secure the stabilization member within the coupling mechanism. The locking device includes an insertion head member, a seating member and a posted member. The posted member has an interface member and at least one post extending therefrom. The seating member is connected to the insertion head member at a break-off junction with at least one hole extending therethrough. The seating member is operatively associated with the coupling mechanism for securing the stabilization member within the coupling mechanism. The at least one post is received and frictionally engages the at least one hole, thereby limiting movement of the posted member relative to the seating member. The at least one post rigidly couples the interface member to the seating member, wherein the posted member is configured for disposition between the seating member and the stabilization member when the locking device is employed to secure the stabilization member within the coupling mechanism. Further, the at least one post connects to the interface member at a break-off junction and allows the interface member to break away and contact the stabilization member with engagement of the seating member to the coupling mechanism. Continued threading of the seating member into the coupling mechanism secures the stabilization member within the coupling mechanism with the posted member disposed between the seating member and the stabilization member. When the stabilization member is secured within the coupling mechanism, the insertion head member may be broken away from the seating member at the break-off junction by applying a pre-selected torque amount to the insertion head member with a torque producing tool.

In a further aspect, a method for stabilizing a spinal column is presented. This method includes: providing a bone stabilization system that includes a bone anchor, a stabilization member, a coupling mechanism, and a locking device, wherein the coupling mechanism is configured to couple the stabilization member to the bone anchor, and the locking device is operatively associated with the coupling mechanism, and wherein the locking device further includes an insertion head member, a seating member and an interface member, wherein the insertion head member is connected to the seating member, and the seating member is connected to the interface member, wherein the seating member operatively engages the coupling mechanism. The method further includes inserting the bone anchor into the coupling mechanism and attaching the bone anchor to a vertebra in the spinal column. The method further includes positioning the stabilization member within the coupling mechanism and engaging the locking device to the coupling mechanism, thereby the seating member threadably engages the coupling mechanism causing the interface member to break away from the seating member and contact the stabilization member. The stabilization member is secured between the interface member and the coupling mechanism by continued threading of the seating member into the coupling mechanism.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 is a perspective view of one embodiment of a bone stabilization system, in accordance with an aspect of the present invention;

FIG. 2 is a perspective view of one embodiment of a coupling mechanism, in accordance with an aspect of the present invention;

FIG. 3 is a cross-sectional elevational view of the coupling mechanism of FIG. 2, taken along line 3-3, in accordance with an aspect of the present invention;

FIG. 4 is a partial elevational view of one embodiment of a bone stabilization system employing a locking device comprising a seating member, an insertion head member and an interface member, shown with the interface member inserted between the arms of the coupling mechanism, in accordance with an aspect of the present invention;

FIG. 5 is a partial elevational view of the bone stabilization system of FIG. 4, showing the seating member operatively engaging the coupling mechanism with the interface member having broken away and contacting the stabilization member, in accordance with an aspect of the present invention;

FIG. 6 is a partial elevational view of the bone stabilization system of FIG. 4, showing the seating member fully engaged in the coupling mechanism following the breaking away of the insertion head member with the interface member securing the stabilization member, in accordance with an aspect of the present invention;

FIG. 7 is a partial elevational view of the bone stabilization system employing another embodiment of a locking device shown with the interface member inserted between the arms of the coupling mechanism, in accordance with an aspect of the present invention;

FIG. 7A is a partial elevational view of the bone stabilization system of FIG. 7, showing the seating member partially engaged in the coupling mechanism following the breaking away of the interface member with the interface member rotatably coupled to the seating member by a post disposed between the interface member and the seating member, in accordance with an aspect of the present invention;

FIG. 7B is a partial elevational view of the bone stabilization system of FIG. 7, showing the seating member fully engaged in the coupling mechanism following breaking away of the insertion head member with the interface member securing the stabilization member, in accordance with an aspect of the present invention;

FIG. 8 is a partial elevational view of one embodiment of a bone stabilization system employing another embodiment of a locking device comprising an insertion head member, a seating member, a post member and an interface member, shown with the interface member with the interface member inserted between the arms of the coupling mechanism, in accordance with an aspect of the present invention;

FIG. 8A is a partial elevational view of the bone stabilization system of FIG. 8, showing the seating member fully engaged in the coupling mechanism following the breaking away of the insertion head member with the interface member securing the stabilization member, in accordance with an aspect of the present invention;

FIG. 9 is an elevational view of one embodiment of a bone stabilization system employing another embodiment of a locking device comprising an insertion head member, a seating member and a posted member, shown with the interface member with the interface member inserted between the arms of the coupling mechanism, in accordance with an aspect of the present invention;

FIG. 9A is a partial elevational view of the bone stabilization system of FIG. 9, showing the seating member operationally engaging the coupling mechanism with the interface member having broken away and contacting the stabilization member, in accordance with an aspect of the present invention;

FIG. 9B is a partial elevational view of the bone stabilization system of FIG. 9, showing the seating member fully engaged in the coupling mechanism following breaking away of the insertion head member with the interface member securing the stabilization member, in accordance with an aspect of the present invention;

FIG. 10A is top plan view of the insertion head member of FIG. 6, in accordance with an aspect of the present invention;

FIG. 10B is a top plan view of a further embodiment of an insertion head member, in accordance with an aspect of the present invention;

FIG. 10C is a top plan view of a further embodiment of an insertion head member, in accordance with an aspect of the present invention;

FIG. 11A is a top plan view of yet another embodiment of a posted member, in accordance with an aspect of the present invention;

FIG. 11B is a top plan view of a further embodiment of a posted member, in accordance with an aspect of the present invention; and

FIG. 11C is a top plan view of a further embodiment of a posted member, in accordance with an aspect of the present invention.

### BEST MODE FOR CARRYING OUT THE INVENTION

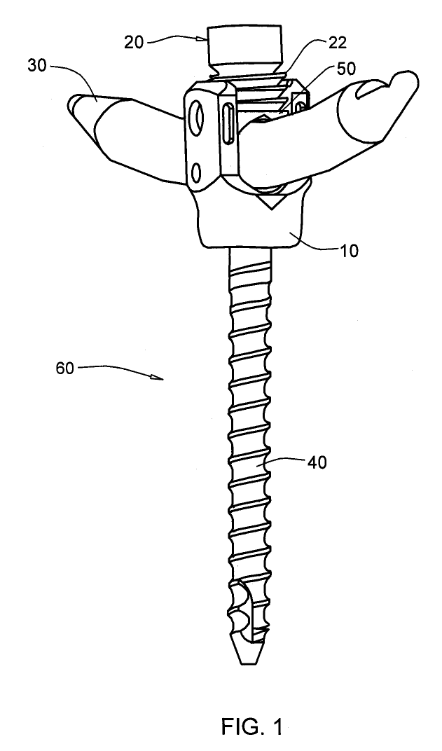

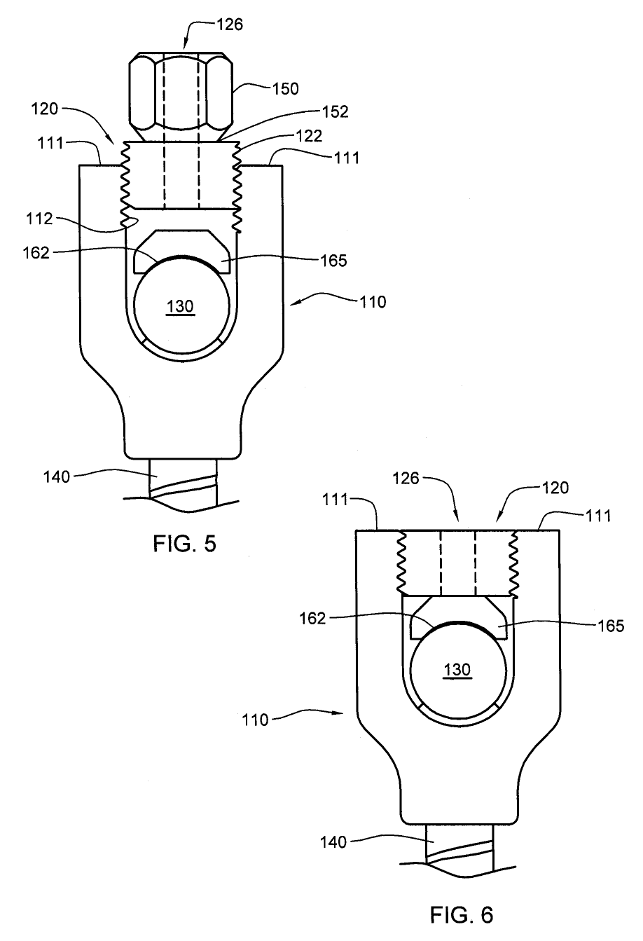

Generally stated, presented herein is an enhanced locking device for a bone stabilization or anchor system and a surgical method for stabilizing a spinal column employing a bone stabilization system and the enhanced locking device. The bone stabilization system includes a bone anchor (e.g., a screw), a coupling mechanism (e.g., an integral tulip head) and a stabilization member (e.g., a rod), wherein the coupling mechanism is configured to couple the stabilization member to the bone anchor. The enhanced locking device includes an insertion head member, seating member (e.g., a threaded locking cap) and an interface member. The insertion head member and the interface member are integrally joined with the seating member, which is operatively associated with the coupling mechanism for securing the stabilization member within the coupling mechanism, and may include at least one opening extending therethrough. The interface member is connected to the seating member for disposition between the seating member and the stabilization member. The interface member may include at least one post for disposition between the seating member and the interface member. The post may extend from the interface member and be received into the at least one opening of the seating member. The converse is also contemplated, wherein the post may extend from the seating member and be received into the at least one opening of the interface member. The at least one post rigidly couples the seating member to the interface member prior to the seating member operatively engaging the coupling mechanism to secure the stabilization member within the coupling mechanism. Various embodiments of the seating member, interface member and post are described below with reference to FIGS. 4-11C. One embodiment of a bone stabilization system is first presented, however, with reference to FIGS. 1-3.

FIG. 1 depicts one embodiment of a bone stabilization system 60, which includes a coupling mechanism 10, a locking device 20, (comprising, in this embodiment, a seating member 22 and a saddle member 50), a stabilization member 30, and a bone anchor 40. When used in a spine to secure multiple levels of the spinal column, each bone anchor 40 is placed within an individual vertebra, and a coupling mechanism 10 is attached to the implanted bone anchor 40. Following placement of multiple bone anchors and coupling mechanisms, an appropriately dimensioned stabilization member 30, which spans one or more levels of the affected vertebral region, is placed within the coupling mechanisms 10 and secured in place employing multiple locking devices. In this initial embodiment, locking device 20 includes seating member 22 and saddle member 50. When the locking device is in use, stabilization member 30 is frictionally held in place between coupling mechanism 10 and seating member 22 by saddle member 50.

In one implementation, the locking device may include a deformable saddle member 50 to reduce the resultant stresses realized in stabilization member 30 by decreasing the generation of surface stress risers when fixed within coupling mechanism 10. This would be achieved by fabricating saddle member 50 from the same or similar material as stabilization member 30 and include a concave distal interface surface that further deforms to the shape of stabilization member 30.

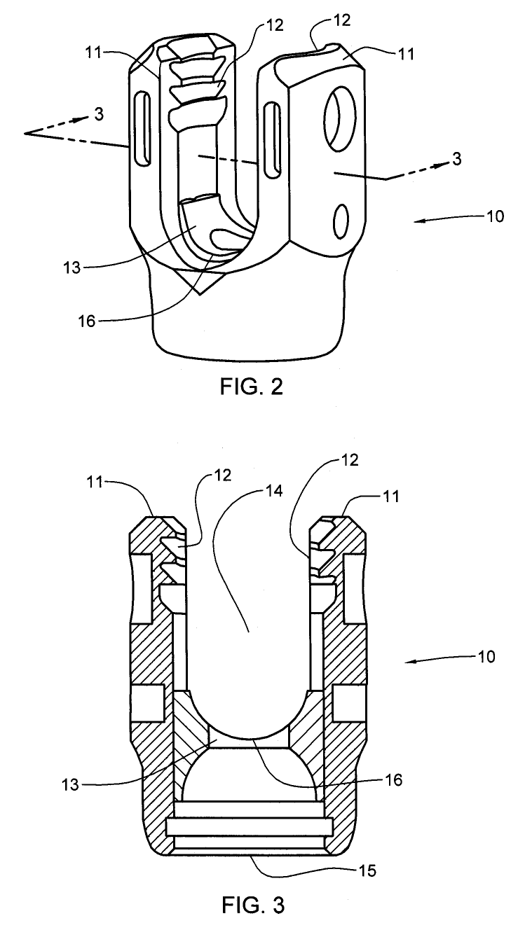

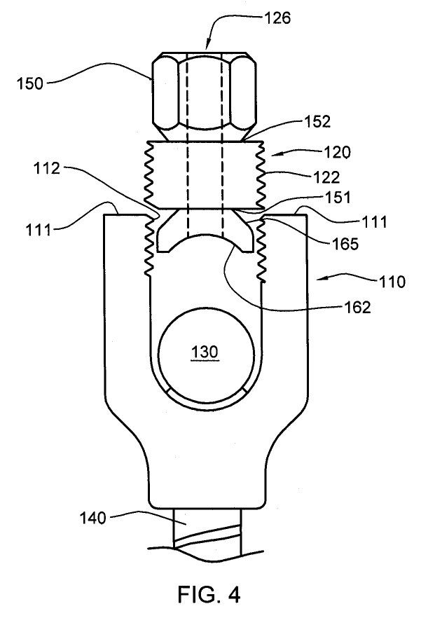

With further reference to FIGS. 2 and 3, coupling mechanism 10 of bone stabilization system 60 includes a channel 14 defined by a seat 13 and a pair of coupling arms 11. Coupling arms 11, which are disposed substantially parallel and project in an upward manner from seat 13, together with seat 13 form a U-shaped channel 14, which is appropriately sized to receive stabilization member 30. The internal walls of coupling arms 11 include internal threads 12 or alternatively an internal cam surface (not shown) to engage external threads of seating member 22. Typically, at least one through hole 15 is located directly below seat 13 in coupling mechanism 10. In one approach, a bone anchor is inserted into hole 15 prior to the placement of the stabilization member. The longitudinal axis of the bone anchor may be at a fixed angle relative to coupling mechanism 10 following insertion into hole 15 or be allowed to pivot within hole 15. Hole 15 may be counter bored, counter sunk, slotted, have a spherical seat, keyed or any combination or derivation of these manufacturing techniques, to allow the top portion of the anchor head to sit below the seat floor 16 and pivot or angle in a single or multiple planar fashion.

In this example, seating member 22 threadably engages with internal threads 12 of coupling mechanism 10, although it should be understood by those skilled in the art that other configurations are possible, including a seating member configured to include an external cam surface (not shown) that engages with an internal cam surface (not shown) located on the internal surface of coupling arms 11. In an unlocked position, stabilization member 30 can move freely within channel 14. When in a locked position, with the seating member 22 substantially engaged with internal threads 12 of coupling mechanism 10, pressure or a compressive force is applied across the distal interface surface of saddle member 50 onto stabilization member 30.

Stabilization member 30 (see FIG. 1) is typically shaped as an elongate and continuous orthopaedic implant, for example, in the shape of a rod. Alternative stabilization members may include, but are not limited to plates, bars, tethers, cables, elastic structures and dynamic stabilization members (not shown). Stabilization member 30 may be fabricated from a plastic material, such as a polyetheretherketone (PEEK) polymer. Alternatively, stabilization member 30 may be fabricated from a material comprising carbon fiber composite polymers, bio-compatible metals, shape memory metals, resorbable polymers, bio-inert polymeric materials, thermoplastic polymers, thermoset polymers or any combination of these materials.

As one detailed example, saddle member 50 may be fabricated from a deformable plastic material, such as polyetheretherketone (PEEK) polymer. Alternatively, saddle member 50 may be fabricated from another deformable material selected from the group consisting of carbon fiber composite polymers, UHMWPE, shape memory metals, low flexural modulus metals, resorbable polymers, bio-inert polymeric materials, thermoplastic polymers, thermoset polymers and any combinations of these materials. In one implementation, the material used to comprise saddle member 50 will have a flexural modulus that is equivalent or similar to the flexural modulus of stabilization member 30. One possible range of the flexural modulus of saddle member 50 is from about 30 to 115 MPa.

Bone anchor 40 is typically configured as a bone screw, although alternative bone anchors may be utilized including bone fixation posts (not shown), bone staples (not shown), hooks (not shown), and moveable multi-axial head or screws (not shown). It should be understood by those skilled in the art that the bone anchor-coupling mechanism structures described herein are presented by way of example only and that other configurations may be used, including coupling mechanism 10 being configured integrally with bone anchor 40.

FIGS. 4 & 5 depict an alternate embodiment of a bone stabilization system, in accordance with an aspect of the present invention. This embodiment is similar to the bone stabilization system embodiment of FIGS. 1-3; however, saddle member 50 of the initial embodiment is replaced by an interface member 165, and the seating member 20 is replaced by a seating member 120 and an insertion head member 150 configured in one embodiment as shown in FIGS. 4 & 5. More particularly, this bone stabilization system includes a coupling mechanism 110, a locking device (comprising seating member 120, insertion head member 150 and an interface member 165), a stabilization member 130, and a bone anchor 140. When in use, bone anchor 140 is placed within an individual vertebra, with a coupling mechanism 110 attached thereto. Coupling mechanism 110 is appropriately dimensioned to receive stabilization member 130, which spans one or more levels of the effected vertebral region. In this embodiment, coupling mechanism 110 again includes two upwardly projecting arms 111 which have (by way of example) threads 112 on an inner surface thereof for threadably receiving seating member 120 of the locking device.

As shown, seating member 120 includes external threads 122 for engagement with coupling mechanism 110 and insertion head member 150 having, in this example, a hexagonal-shaped perimeter. A central cannulation or opening 126 may extend through insertion head member 150, seating member 120 and interface member 165. In the embodiment illustrated in FIGS. 4 & 5, interface member 165 may be integrally connected to one end of seating member 120 at a first break-off junction 151 with the second end of seating member 120 being integrally connected to insertion head member 150 at a second break-off junction 152. The distal interface surface 162 of interface member 165 is saddle-style contoured to facilitate physical contact with stabilization member 130.

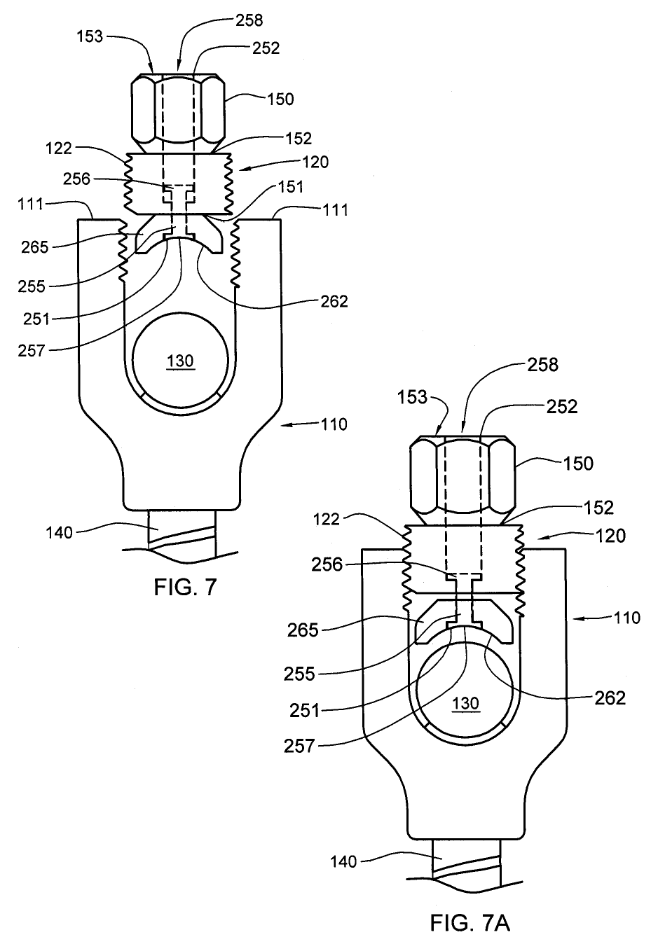

As shown in FIG. 5, in operation, interface member 165 breaks away from seating member 120 at second break-off junction 152 upon the engagement of seating member 120 into coupling mechanism 110. The torque force required for interface member 165 to break-off from seating member 120 is less than the torque force necessary for breaking away insertion head member 150. Upon break-off, interface member 165 drops within coupling mechanism 110 making contact with a top exterior portion of stabilization member 130. Continued threading of seating member 120 into coupling mechanism 110 results in seating member 120 pressingly engaging interface member 165 to further contact stabilization member 130, and thereby secure stabilization member 130 within coupling mechanism 110.

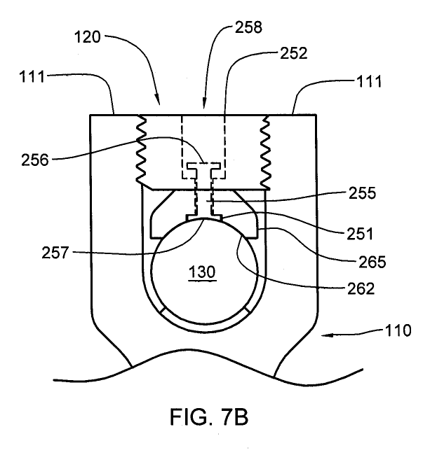

FIG. 6 shows seating member 120 fully engaged with coupling mechanism 110 resulting in distal interface surface 162 contacting the top exterior portion of stabilization member 130 and securing stabilization member 130 between interface member 165 and coupling mechanism 110. Insertion head member 150 has broken away from seating member 120 at second break-off junction 152 following the application of a pre-selected maximum torque force by a torque producing insertion surgical tool (not shown). Considerations for the determination of this maximum torque value include the type of material employed for the locking device, the diameter of the break-off junction, as well as the amount of material circumferentially removed from insertion head member 150 to define break-off junction 152. The maximum torque force range necessary to cause insertion head member 150 to break away from seating member 120 is from about 9 N·M to about 13 N·M. The maximum torque value to cause interface member 165 to break away from seating member 120 is preferably less than 9 N·M. A low profile surface results following the breaking away of insertion head member 150. The low profile implant surface is advantageous in that such a construct decreases the potential for post-operative complications, including soft-tissue irritation around the spinal column.

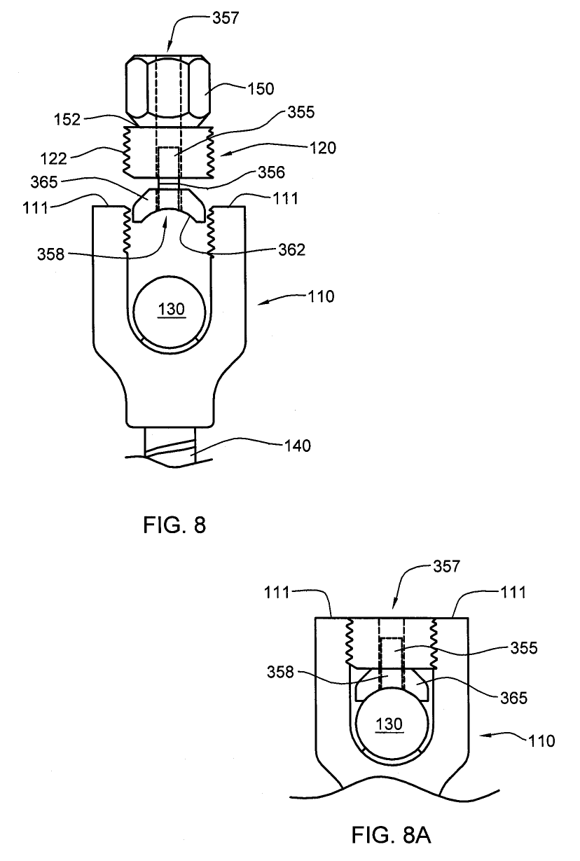

FIGS. 7, 7A & 7B depict an alternate embodiment of a locking device for a bone stabilization system, which again includes a bone anchor 140, a stabilization member 130 and a coupling mechanism 110 configured to couple the stabilization member to the bone anchor by cradling the stabilization member as shown. Stabilization member 130 may extend through any number of coupling mechanism-bone anchor assemblies. In this embodiment, the locking device comprises a seating member 120 and an insertion head member 165 such as described above in connection with FIGS. 4 & 5, and an alternate embodiment of an interface member 265. This alternate embodiment includes interface member 265 and a cylindrical post 255. Post 255 includes two radially extending flare portions 256, 257 locate proximate to the proximal and distal ends of post 255. As described above in connection with FIGS. 4 & 5, interface member 265 may be integrally connected to one end of seating member 120 at a first break-off junction 151 with the second end of seating member 120 being integrally connected to insertion head member 150 at a second break-off junction 152. A central hole 258 extends axially through insertion head member 150 and seating member 120 with central hole 258 preferably having a first counterbore 252 relative to the top surface 153 of insertion head member 150 and a second counterbore 251 preferably relative to the distal interface surface 262. The assembly process for the retention of post 255 within central hole 258 may include the initial step of applying a deforming load to one end of post 255, thereby forming a first radially extending flare portion 256 proximate to the first end. Preferably, post 255 is inserted into hole 258 allowing flare portion 256 to contact the internal shoulder of first counterbore 252. Following insertion of post 255 into hole 258, post 255 is disposed between seating member 120 and interface member 265. Typically, a deforming load is then applied to the now distal or second end of post 255, thereby forming a second radially extending flare portion 257 proximate to the distal end of post 255. Flare portion 257 contacts the internal shoulder of counterbore 251 and secures post 255 within hole 258. The distal interface surface 262 of interface member 265 is again saddle-style contoured to physically engage a portion of the outer surface of stabilization member 130.

In operation as shown in FIG. 7A, interface member 265 is placed within the coupling arms 111 of coupling mechanism 110 allowing seating member 120 to threadably engage coupling mechanism 110. Engagement of seating member 120 causes interface member 265 to break away from seating member 120 at break-off junction 151. Post 255 acts to hold and allow seating member 120 to be rotatably coupled to interface member 265 within coupling mechanism 110. Continued threading of seating member 120 into coupling mechanism 110 results in seating member 120 pressingly engaging interface member 265 to further contact stabilization member 130 and, thereby securing stabilization member 130 within coupling mechanism 110. Post 255 remains positioned within hole 258, with counterbore 251 being configured and dimensioned to provide for flare portion 257 to remain below distal interface surface 262 when seating member 120 is fully engaged and applying a load that frictionally secures stabilization member 130 within coupling mechanism 110.

FIG. 7B shows seating member 120 fully engaged with coupling mechanism 110 resulting in distal interface surface 262 contacting the top exterior portion of stabilization member 130 and securing stabilization member 130 between interface member 265 and coupling mechanism 110. As described above in connection with FIGS. 4 & 5, insertion head member 150 breaks away from seating member 120 at the second break-off junction 152 following the application of a pre-selected maximum torque force (in the range described in connection with FIGS. 5 & 6 above) by an insertion surgical tool (not shown) producing an advantageous low profile implant surface.

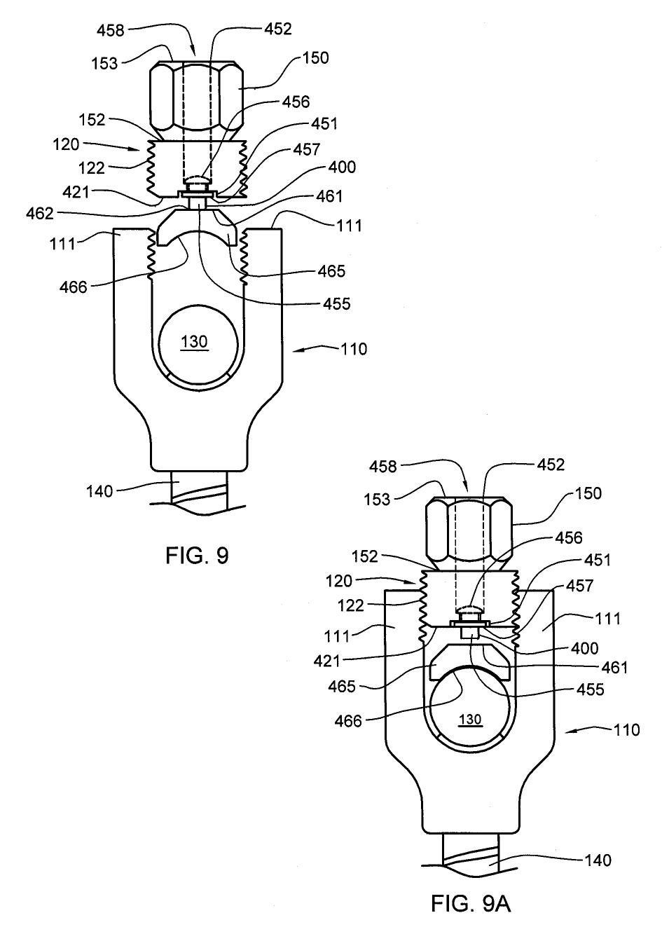

FIG. 8 depicts an alternate embodiment of a locking device for a bone stabilization system, which again includes a bone anchor 140, a stabilization member 130 and a coupling mechanism 110. In this embodiment, the locking device comprises a seating member 120 and insertion head member 150 such as described above in connection with FIGS. 4 & 5, and an alternate embodiment of an interface member 365. This alternate embodiment includes interface member 365 and a post member 355. Post member 355 is configured with a circumferential break-off line 356 disposed around the perimeter of the post and intermediate the ends thereof. This break-off line can be formed, for example, by circumferentially removing material from post member 355 at the desired location. Preferably, the cross-section shape of post member 355 is square, rectangular, triangular, hexagonal or other non-circular geometric shape. Seating member 120 and insertion head member 150 are integrally connected and include a centrally located axial hole 357 extending completely therethrough. Preferably, interface member 365 also includes a centrally located through hole 358. The assembly process of this alternative embodiment of the locking device includes inserting the two ends of post member 355 into the corresponding central holes 357, 358 of seating member 120 and interface member 365. When inserted, the ends of post member 355 are frictionally engaged within central holes 357, 358 resulting in the rigid coupling of seating member 120 to interface member 365. As described above in connection with FIGS. 4 & 5, seating member 120 is integrally connected to insertion head member 150 at a break-off junction 152. The distal interface surface 362 of interface member 365 is again contoured to physically engage a portion of the outer surface of stabilization member 130.

This alternative locking device embodiment operates in a manner similar to the embodiment shown in FIG. 7 above, in that interface member 365 is placed within the coupling arms 111 of coupling mechanism 110 allowing seating member 120 to threadably engage coupling mechanism 110. Engagement of seating member 120 causes post member 335 to break at circumferential break-off line 356 allowing interface member 365 to drop within coupling mechanism 110 and make contact with a top exterior portion of stabilization member 130. Continued threading of seating member 120 into coupling mechanism 110 results in seating member 120 pressingly engaging interface member 365 to further contact stabilization member 130 and secure stabilization member 130 within coupling mechanism 110.

As shown in FIG. 8A, this alternative locking device embodiment operates in a manner similar to the embodiment shown in FIG. 7A above in that following full engagement of seating head 120 and securement of stabilization member 130, insertion head member 150 may be broken away from seating member 120 at break-off junction 152. Such break-off will occur when a pre-selected maximum torque force is applied by a torque producing insertion surgical tool (not shown) to insertion head member 150 in the range described in connection with FIGS. 5 & 6 above.

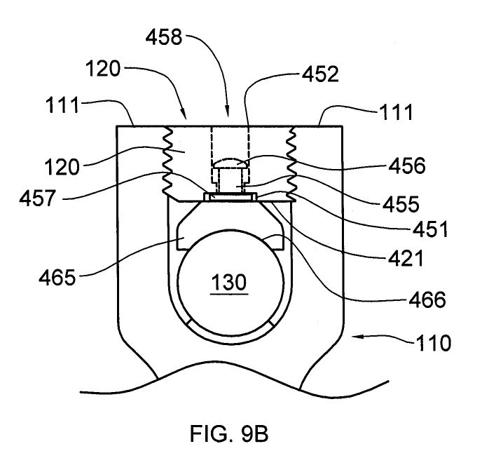

FIGS. 9, 9A & 9B depict a further alternate embodiment of a locking device for a bone stabilization system, which again includes a bone anchor 140, a stabilization member 130 and a coupling mechanism 110 configured to couple the stabilization member to the bone anchor by cradling the stabilization member as shown. In this embodiment, the locking device comprises a seating member 120 such as described above in connection with FIGS. 4 & 5, and an alternate embodiment of a posted member 400. In this alternate embodiment, posted member 400 includes an interface member 465 and a post 455 extending from a proximal surface 461 thereof. The distal interface surface 466 of interface member 465 is again saddle-style contoured to physically engage a portion of the outer surface of stabilization member 130. Post 455 includes a first break-off junction 462 proximate to the interface between post 455 and interface member 465 and a circumferential flange 457 or snap ring disposed intermediate the end of post 455 and the break-off junction 462. Post 455 further includes one radially extending flare portion 456 located proximate to the end of post 455. As described above in connection with FIGS. 7 & 7A, seating member 120 is integrally connected to insertion head member 150 at a second break-off junction 152. Preferably, central hole 458 extends axially through insertion head member 150 and seating member 120 with central hole 458 typically having a first counterbore 452 relative to a top surface 153 of insertion head member 150 and preferably, a second counterbore 451 relative to a bottom surface 421 of seating member 120. The preferred assembly process for coupling interface member 465 to seating member 120 includes the step of inserting the end of post 455 into central hole 458 of seating member 120. Following the insertion step, a deforming load is applied to the end of post 455, thereby forming a radially extending flare portion 456 proximate to the end of post 455. Preferably, flare portion 456 contacts the internal shoulder of first counterbore 452 and circumferential flange 457 contacts the internal shoulder of second counterbore 451. Following insertion of post 455 into hole 458, posted member 400 is rigidly coupled to seating member 120.

In operation, this alternative locking device embodiment functions in a manner similar to the embodiment shown in FIG. 8 above, in that posted member 400 is placed within the coupling arms 111 of coupling mechanism 110 allowing seating member 120 to threadably engage coupling mechanism 110. As shown in FIG. 9A, engagement of seating member 120 causes post 455 to break at first break-off junction 462 allowing interface member 465 to drop within coupling mechanism 110 and make contact with a top exterior portion of stabilization member 130. The torque force required for post 455 to break off is usually less than the torque force necessary for insertion head member 150 to break away from seating member 120. Continued threading of seating member 120 into coupling mechanism 110 results in post 455 pressingly engaging interface member 465 to further contact stabilization member 130 and secure stabilization member 130 within coupling mechanism 110.

As shown in FIG. 9B, this alternative locking device embodiment operates in a manner similar to the embodiment shown in FIG. 8A above in that following full engagement of seating member 120 and securement of stabilization member 130 within coupling mechanism 110, insertion head member 150 may be broken away from seating member 120 at second break-off junction 152. Such break-off will occur when a pre-selected maximum torque force is applied to insertion head member 150 by a torque producing insertion surgical tool (not shown). The range of the maximum torque force being from about 9 N·M to about 13 N·M.

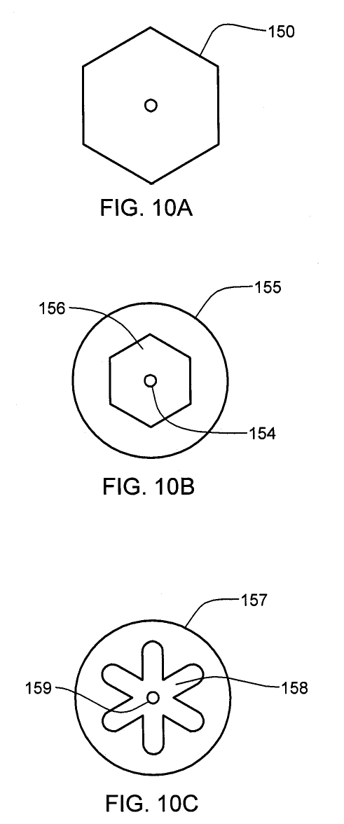

FIG. 10A depicts a further embodiment of an insertion head member for a locking device, in accordance with an aspect of the present invention. As shown, insertion head member 150 is configured with an outer hexagonal-shaped perimeter. The outer perimeter of insertion head member 150 allows for the multi-surface securement of a torque producing insertion surgical tool. Alternatively, FIG. 10B shows a top plan view of insertion head member 155 that preferably includes an internal hexagonal-shaped opening 156 extending therein. The hexagonal-shaped opening 156 in this example is aligned with a central axially extending opening 154. The insertion head member 155 may be configured with a non-hexagonal-shaped outer perimeter. FIG. 10C depicts a top plan view of a further alternate embodiment of an insertion head member 157. In this embodiment, the top surface of insertion head member 157 has an internal hexalobular-shaped opening 158 for engagement with a torque producing insertion surgical tool (not shown). As in the embodiment of FIG. 10B, the insertion head member 157 may be configured with a non-hexagonal-shaped outer perimeter and include a central axial extending opening 159 aligned with internal hexalobular-shaped opening 158 to allow for the insertion of a construct structure (e.g., a post or pin) or a securement fixture for aligning a torque producing insertion surgical tool (not shown). The above-described examples of the shapes of the internal openings of insertion head member 155 are not exclusive. Alternate-shaped embodiments of the internal openings are contemplated, including rectangular, square, triangular or other polygonal shapes.

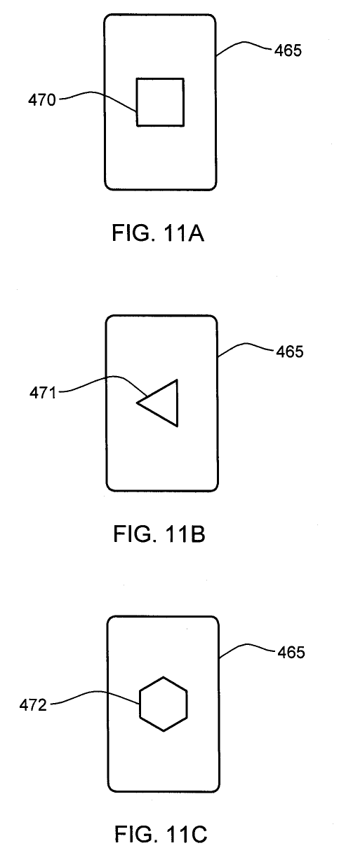

FIGS. 11A, 11B and 11C depict further geometric post variations for posted member 400. In the top plan view of FIG. 11A, the posted member is shown to comprise an interface member 465 from which a square post 470 projects. In this example, post 470 is again an elongate post extending from interface member 465 in a manner similar to the post described above. However, in this embodiment, post 470 has a square transverse cross-section as shown in the plan view. Any desired geometric configuration can be employed for the post. FIGS. 11B & 11C depict alternate embodiments, wherein a triangular-shaped post 471 and a hexagonal-shaped post 472 respectively, extend from interface member 465. Other transverse cross-sections for post 470 could include rectangular, oblong, etc.

The locking device (seating member 120, insertion head member 150 and interface member 165) may be fabricated from a titanium alloy, for example, the alloy Ti-6A1-4V. Alternatively, the locking device may be fabricated from one or more of CP titanium, cobalt-chromium, a 300 series stainless steel, carbon fiber materials, carbon fiber composites, resorbable polymers, bio-inert polymeric materials, thermoplastic polymers, thermoset polymers, or any combination of these materials. Additionally, interface member 165 may also be fabricated from a different biocompatible material as listed above. For example, interface member 165 may be fabricated of a material which elastically deforms, and thereby fixedly secures stabilization member 130 when the locking device is threadably advanced into coupling mechanism 110. By way of example, interface member 165 could be formed from a deformable plastic material, such as polyetheretherketone (PEEK) polymer. Alternatively, interface member 165 could be fabricated from another deformable material comprising carbon fiber composite polymers, UHMWPE, shape memory metals, resorbable polymers, bio-inert polymeric materials, thermoplastic polymers, thermoset polymers, or any combination of these materials.

In view of the above description, those skilled in the art will note that a method for stabilizing a spinal column is presented herein. This method includes: providing a bone stabilization system comprising a bone anchor, a stabilization member, a coupling mechanism, and a locking device, wherein the coupling mechanism is configured to couple the stabilization member to the bone anchor, and the locking device is operatively associated with the coupling mechanism, and wherein the locking device further includes a seating member configured to threadably engage the coupling mechanism, an insertion head member, and an interface member, wherein the insertion head member is integrally connected to the seating member, and wherein the interface member includes a distal interface surface, wherein the distal interface surface comprises one of a planar surface or a contoured surface, the contoured surface being contoured to mate with the stabilization member; inserting the bone anchor into the coupling mechanism and attaching the bone anchor to a vertebra within the spinal column; positioning the stabilization member in the coupling mechanism; engaging the locking device to the coupling mechanism; threading the seating member into the coupling device causing the interface member to break away from the seating member, and thereby allowing the interface member to contact the stabilization member; securing the stabilization member between the interface member and the coupling mechanism by continuing to threadably advance the seating member into the coupling mechanism.

The method can further include breaking off the insertion head member from the seating member at a break-off junction by applying a pre-selected torque force to the insertion head member with a torque producing tool.

To summarize, those skilled in the art will note from the above description that provided herein is an enhanced locking device for a bone stabilization system and surgical methods for stabilizing a column employing a bone stabilization system and the enhanced locking device. The bone stabilization system includes a bone anchor, a coupling mechanism, and a stabilization member, wherein the coupling mechanism is configured to couple the stabilization member to the bone anchor. The enhanced locking device includes a seating member, an insertion head member and an interface member. The seating member is operatively associated with the coupling mechanism for securing a stabilization member within the coupling mechanism, and includes at least one opening therein. The insertion head member is connected to the seating member at a break-away junction. The interface member is connected directly to the seating member or alternatively, may be coupled together by a rigid post. The interface member is configured for disposition between the seating member and the stabilization member when the seating member is employed to secure the stabilization member within the coupling mechanism.

Advantageously, allowing the insertion head to break away from the seating member results in a low profile spinal implant. Further, rigid coupling of the interface member to the seating member and the interface member being configured to break-away when the seating member engages the coupling mechanism facilitates alignment and securement of the stabilization member. Numerous variations on the seating member and interface member and the methods of coupling these two structures are depicted and described herein.

By way of example, the distal surface of the interface member can comprise a number of different geometries, including planar and saddle-style contoured. A saddle-style contoured geometry that follows the outer periphery of the stabilization member is beneficial for semi-rigid stabilization members because the surface allows the force on the stabilization member to be distributed across the entire geometry. Those skilled in the art will note, however, that the geometric shape of the distal interface surface of the interface member is not limited to the surfaces described herein. Further, the outer profile of the interface member is not constrained to being enclosed by the perimeter of the coupling mechanism. That is, the interface member may extend past the arms of the coupling mechanism.

Although the preferred embodiments have been depicted and described in detail herein, it will be apparent to those skilled in the relevant art that various modifications, additions and substitutions can be made without departing from its essence and therefore these are to be considered to be within the scope of the following claims.

What is claimed is:

1. A locking device for use in a bone stabilization system, the bone stabilization system including a bone anchor, a coupling mechanism and a stabilization member, wherein the coupling mechanism is configured to couple the stabilization member to the bone anchor, the locking device comprising:
an insertion head member;
a seating member rigidly connected to the insertion head member, wherein the seating member is configured to engage the coupling mechanism for securing the stabilization member within the coupling mechanism; and
an interface member coupled to the seating member, wherein the seating member is configured to engage the coupling mechanism when the locking device is employed to secure the stabilization member within the coupling mechanism.

2. The locking device of claim 1, wherein the interface member connects to the seating member at a break-off junction.

3. The locking device of claim 2, wherein the interface member breaks away from the seating member at the break-off junction with engagement of the seating member to the coupling mechanism, thereby allowing the interface member to contact the stabilization member.

4. The locking device of claim 2, wherein the seating member is configured to threadably engage the coupling mechanism, and wherein threading of the seating member into the coupling mechanism results in the interface member breaking away from the seating member at the break-off junction, thereby allowing the seating member to contact the stabilization member, and wherein continued threading of the seating member into the coupling mechanism results in securing the stabilization member between the interface member and the coupling mechanism.

5. The locking device of claim 1, wherein the insertion head member connects to the seating member at a break-off junction.

6. The locking device of claim 5, wherein the insertion head member breaks away from the seating member at the break-off junction when a pre-selected torque force is applied to the insertion head member.

7. The locking device of claim 1, wherein the insertion head member is configured to facilitate coupling thereto by an insertion tool, wherein the insertion tool when operative produces a torque on the insertion head member, and wherein the insertion head member comprises one of an outer hexagonal-shaped perimeter, an internal hexagonal-shaped opening, an internal polygonal-shaped opening or an internal hexalobular-shaped opening.

8. The locking device of claim 1, wherein the stabilization member is an elongate orthopaedic implant having a first end, a second end and a longitudinal axis extending therebetween, and wherein when the locking device is in use the stabilization member is received within the coupling mechanism and the interface member engages the stabilization member along the longitudinal axis thereof.

9. The locking device of claim 8, wherein the interface member includes a distal interface surface, wherein the distal interface surface comprises one of a planar surface or a contoured surface, the contoured surface being contoured to mate to an exterior portion of the stabilization member when the seating member is fully engaged within the coupling mechanism, thereby securing the stabilization member between the coupling mechanism and the interface member.

10. The locking device of claim 1, wherein the insertion head member, the seating member and the interface member are fabricated from a biocompatible metal.

11. The locking device of claim 1, wherein the locking device further comprises at least one post configured for disposition between the seating member and the interface member with the at least one post being partially received within at least one hole of the seating member and being partially received within at least one hole of the interface member.

12. The locking device of claim 11, wherein the at least one post comprises an elongate post having a first end and a second end, wherein proximate to the first end is a first flare portion which extends radially therefrom, and proximate to the second end is a second flare portion which extends radially therefrom.

13. The locking device of claim 12, wherein the insertion head member and the seating member are integrally joined and at least one hole extends axially therethrough, wherein the at least one hole is counterbored relative to a top surface of the insertion head member to provide a first internal shoulder sized to contact the first flare portion of the post, and wherein the at least one hole is further counterbored relative to the distal interface surface of the interface member to provide a second internal shoulder sized to contact the second radially extending flare portion of the at least one post.

14. The locking device of claim 13, wherein the interface member and the seating member are rotatably coupled to each other via the at least one post.

15. A locking device for use in a bone stabilization system, the bone stabilization system including a bone anchor, a coupling mechanism and a stabilization member, wherein the coupling mechanism is configured to couple the stabilization member to the bone anchor, the locking device comprising:
an insertion head member;
a post member;
a seating member connected to the insertion head member, wherein the seating member is configured to engage the coupling mechanism for securing the stabilization member within the coupling mechanism, the seating member being configured with at least one opening therein for receiving a first end of the post member;
an interface member with at least one opening therein for receiving a second end of the post member; and
wherein the post member is disposed between the seating member and the interface member, thereby coupling the seating member to the interface member.

16. The locking device of claim 15, wherein the post member extends longitudinally between the first end to the second end, and comprises a transverse cross-section comprising one of a square cross-section, a triangular cross-section, a rectangular cross-section, an oblong cross-section, or a hexagonal cross-section.

17. The locking device of claim 15, wherein the at least one opening in the seating member comprises a central opening configured to receive and frictionally engage the first end of the post member, and wherein the at least one opening in the interface member comprises a central opening configured to receive and frictionally engage the second end of the post member.

18. The locking device of claim 17, wherein the post member rigidly couples the seating member to the interface member.

19. The locking device of claim 15, wherein a circumferential break-off line extends around the perimeter of the post member, and wherein the circumferential break-off line is disposed intermediate the first end and the second end of the post member.

20. The locking device of claim 19, wherein the interface member breaks away from the seating member at the circumferential break-off line with the engagement of the seating member to the coupling mechanism, thereby allowing the interface member to contact the stabilization member.

21. The locking device of claim 19, wherein the seating member is configured to threadably engage the coupling mechanism, wherein threading of the seating member into the coupling mechanism results in the post member breaking at the circumferential break-off line, thereby allowing the seating member to contact the stabilization member, wherein continued threading of the seating member into the coupling mechanism results in securing the stabilization member between the interface member and the coupling mechanism.

22. The locking device of claim 15, wherein the insertion head member connects to the seating member at a break-off junction, and wherein the insertion head member breaks away from the seating member at the break-off junction when a pre-selected torque force is applied to the insertion head member.

23. The locking device of claim 22, wherein the insertion head member is configured to facilitate coupling thereto by an insertion tool, wherein the insertion tool when operative produces a torque on the insertion head member, and wherein the insertion head member comprises one of an outer hexagonal-shaped perimeter, an internal hexagonal-shaped opening, an internal polygonal-shaped opening or an internal hexalobular-shaped opening.

24. The locking device of claim 15, wherein the stabilization member is an elongate orthopaedic implant having a first end, a second end and a longitudinal axis extending therebetween, and wherein when the locking device is in use the stabilization member is received within the coupling mechanism and the interface member engages the stabilization member along the longitudinal axis thereof.

25. The locking device of claim 24, wherein the interface member includes a distal interface surface, wherein the distal interface surface comprises one of a planar surface or a contoured surface, the contoured surface being contoured to mate to an exterior portion of the stabilization member when the seating member is fully engaged within the coupling mechanism, thereby securing the stabilization member between the coupling mechanism and the interface member.

26. The locking device of claim 15, wherein the insertion head member, the seating member and the interface member are fabricated from a biocompatible metal.

27. A locking device for use in a bone stabilization system, the bone stabilization system including a bone anchor, a coupling mechanism and a stabilization member, wherein the coupling mechanism is configured to couple the stabilization member to the bone anchor, the locking device comprising:
an insertion head member;
a seating member connected to the insertion head member, wherein the seating member is configured to engage the coupling mechanism for securing the stabilization member within the coupling mechanism, the seating member being configured with at least one opening therein;
a posted member comprising an interface member and at least one post extending therefrom, the posted member configured for disposition between the seating member and the stabilization member with the at least one post extending from the interface member and being partially received into the at least one opening of the seating member; and
wherein the at least one post rigidly couples the interface member to the seating member.

28. The locking device of claim 27, wherein the at least one post has a first end and a second end, and wherein the first end of the at least one post is integrally connected to the interface member, the second end of the at least one post comprises a flare portion radially extending therefrom, and wherein a radially extending circumferential flange is disposed intermediate the first and second end of the at least one post, and wherein the flare portion and the circumferential flange contact the seating member to rigidly couple the interface member to the seating member.

29. The locking device of claim 28, wherein the insertion head member and the seating member are integrally joined and at least one hole extends axially therethrough, wherein the at least one hole is counterbored relative to a top surface of the insertion head member to provide a first internal shoulder sized to contact the flare portion of the second end of the at least one post, and wherein the at least one hole is counterbored relative to a bottom surface of the seating member to provide a second internal shoulder sized to contact the radially extending circumferential flange of the at least one post, wherein the radially flare portion and the circumferential flange thereby limit movement of the posted member relative to the seating member.

30. The locking device of claim 28, wherein the first end of the at least one post connects to the interface member at a break-off junction, and wherein the interface member breaks away from the at least one post at the break-off junction with engagement of the seating member to the coupling mechanism, thereby allowing the interface member to contact the stabilization member.

31. The locking device of claim 30, wherein the seating member is configured to threadably engage the coupling mechanism, wherein threading of the seating member into the coupling mechanism results in the interface member breaking away from the seating member at the break-off junction, thereby allowing the seating member to contact the stabilization member, wherein continued threading of the seating member into the coupling mechanism results in securing the stabilization member between the interface member and the coupling mechanism.

32. The locking device of claim 27, wherein the insertion head member connects to the seating member at a break-off junction, and wherein the insertion head member breaks away from the seating member at the break-off junction when a pre-selected torque force is applied to the insertion head member.

33. The locking device of claim 32, wherein the insertion head member is configured to facilitate coupling thereto by an insertion tool, wherein the insertion tool when operative produces a torque on the insertion head member, and wherein the insertion head member comprises one of an outer hexagonal-shaped perimeter, an internal hexagonal-shaped opening, a polygonal-shaped opening or an internal hexalobular-shaped opening.

34. The locking device of claim 27, wherein the stabilization member is an elongate orthopaedic implant having a first end, a second end and a longitudinal axis extending therebetween, and wherein when the locking device is in use the stabilization member is received within the coupling mechanism and the interface member engages the stabilization member along the longitudinal axis thereof.

35. The locking device of claim 27, wherein the interface member includes a distal interface surface, wherein the distal interface comprises one of a planar surface or a contoured surface, the contoured surface being contoured to mate to an exterior portion of the stabilization member when the seating member is fully engaged within the coupling mechanism, thereby securing the stabilization member between the coupling mechanism and the interface member.

36. The locking device of claim 28, wherein the radially extending circumferential flange is a snap ring.

37. The locking device of claim 31, wherein the insertion head member, the seating member and the interface member are fabricated from a biocompatible metal.

38. A bone stabilization system comprising:
a bone anchor;
a stabilization member;
a coupling mechanism, wherein the coupling mechanism is configured to operatively connect the stabilization member to the bone anchor; and
a locking device, wherein the locking device operatively connects to the coupling mechanism to secure the stabilization member within the coupling mechanism, and wherein the locking device comprises:
an insertion head member;
a seating member connected to the insertion head member, wherein the seating member is configured to engage the coupling mechanism for securing the stabilization member within the coupling mechanism; and
an interface member rigidly coupled to the seating member, wherein the interface member is configured to contact the stabilization member when the seating member is engaged with the coupling mechanism.

39. The bone stabilization system of claim 38, wherein the locking device further comprises at least one post configured for disposition between the seating member and the interface member, and wherein the at least one post comprises a first end and a second end, the first end of the at least one post being coupled to the seating member, and the second end being coupled to the interface member, and wherein the at least one post rotatably couples the seating member to the interface member.

40. The bone stabilization system of claim 38, wherein the locking device is further comprised of a post member, wherein the post member extends longitudinally between a first end and a second end, and comprises a transverse cross-section comprising one of a square cross-section, a triangular cross-section, a rectangular cross-section, an oblong cross-section or a hexagonal cross-section, and wherein the seating member being configured with at least one central opening receives and frictionally engages the first end of the post member, and wherein the interface member being configured with at least one central opening receives and frictionally engages the second end of the post member, and wherein the post member rigidly couples the seating member to the interface member.

41. The bone stabilization system of claim 40, wherein a circumferential break-off line extends around the perimeter of the post member, and wherein the circumferential break-off line is disposed intermediate the first end and the second end of the post member, and wherein the interface member breaks away from the seating member at the circumferential break-off line with the engagement of the seating member to the coupling mechanism, thereby allowing the interface member to contact the stabilization member.

42. The bone stabilization system of claim 38, wherein the locking device further comprises a posted member, wherein the posted member comprises an interface member and at least one post extending therefrom, and wherein a first end of the at least one post is integrally connected to the interface member at a breakaway joint, and a second end of the at least one post comprises a flare portion radially extending therefrom and wherein, a radially extending circumferential flange is disposed intermediate the first and second end of the at least one post, and wherein the flare portion and circumferential flange contact the seating member to rigidly couple the interface member to the seating member.

43. The bone stabilization system of claim 42, wherein the insertion head member and the seating member are integrally joined and at least one hole extends axially therethrough, wherein the at least one hole is counterbored relative to a top surface of the insertion head member to provide a first internal shoulder sized to contact the flare portion of the second end of the at least one post, and wherein the at least one hole is counterbored relative to a bottom surface of the seating member to provide a second internal shoulder sized to contact the radially extending circumferential flange of the at least one post, wherein the radially flare portion and the circumferential flange, thereby limit movement of the posted member relative to the seating member.

44. The bone stabilization system of claim 43, wherein the first end of the at least one post connects to the interface member at a break-off junction, and wherein the interface member breaks away from the at least one post at the break-off junction with engagement of the seating member to the coupling mechanism, thereby allowing the interface member to contact the stabilization member.

45. The bone stabilization system of claim 38, wherein the insertion head member connects to the seating member at a break-off junction, and wherein the insertion head member breaks away from the seating member at the break-off junction when a pre-selected torque force is applied to the insertion head member by an insertion tool.

46. The bone stabilization system of claim 38, wherein the stabilization member is an elongate orthopaedic implant having a first end, a second end and a longitudinal axis extending therebetween, and wherein when the locking device is in use the stabilization member is received within the coupling mechanism and the interface member engages the stabilization member along the longitudinal axis thereof.

47. The bone stabilization system of claim 46, wherein the interface member includes a distal interface surface, wherein the distal interface surface comprises one of a planar surface or a contoured surface, the contoured surface being contoured to mate to an exterior portion of the stabilization member when the seating member is fully engaged within the coupling mechanism, thereby securing the stabilization member between the coupling mechanism and the interface member.

48. The bone stabilization system of claim 38, wherein the insertion head member, the seating member and the interface member are fabricated from a biocompatible metal.

49. A method for stabilizing a spinal column, the method comprising:
providing a bone stabilization system comprising a bone anchor, a stabilization member, a coupling mechanism, and a locking device, wherein the coupling mechanism is configured to couple the stabilization member to the bone anchor, and the locking device is operatively associated with the coupling mechanism, and wherein the locking device further comprises an insertion head member, a seating member and an interface member, wherein the insertion head member is connected to the seating member, and wherein the interface member includes a distal interface surface, wherein the distal interface surface comprises one of a planar surface or a contoured surface, the contoured surface being contoured to contact with the stabilization member;
inserting the bone anchor into the coupling mechanism;
attaching the bone anchor to a vertebra within the spinal column;
positioning the stabilization member in the coupling mechanism;
engaging the locking device to the coupling mechanism;
threadably engaging the seating member into the coupling mechanism, thereby causing the interface member to breakaway from the seating member and allowing the interface member to contact the stabilization member; and
securing the stabilization member between the interface member and the coupling mechanism by continued threading of the seating member into the coupling mechanism.

50. The method of claim 49, wherein the providing further comprises providing the insertion head member being connected to the seating member at a break-off junction.

51. The method of claim 50, wherein the providing further comprises breaking away of the insertion head member from the seating member at the break-off junction when a pre-selected torque force is applied to the insertion head member by a torque producing tool.
